(19) United States
(12) Patent Application Publication
(10) Pub. No.: US 2005/0089891 A1
(43) Pub. Date: Apr. 28, 2005

(54) High throughput beta-globin genotyping method by multiplexed melting temperature analysis

(76) Inventors: Zhili Lin, Sewickley, PA (US); Joseph G. Suzow, Monroeville, PA (US); Jamie M. Fontaine, Cranberry Township, PA (US)

Correspondence Address: MCKAY & ASSOCIATES, PC., 801 MCNEILLY ROAD, PITTSBURG, PA, 15226, US

(21) Appl. No.: 10/885,253

(22) Filed: Jul. 6, 2004

Related U.S. Application Data
(60) Provisional application No. 60/514,166, filed on Oct. 24, 2003.

(51) Int. Cl. C12Q 1/68; C12P 19/34

(52) U.S. Cl. 435/6; 435/91.2

(57) ABSTRACT

What is disclosed is a system and method utilizing an automation system for high throughput DNA extraction and PCR setup, a conventional thermal cycler, and a LIGHTTYPER™ instrument for post-PCR melting temperature analysis for beta-globin mutations. Melting temperature analysis is achieved through fluorescent resonance energy transfer (FRET) reaction using the LIGHTTYPER™ instrument. The assay is designed to simultaneously detect three common beta-globin mutations, S(A173T), C(G172A), and E(G232A), and can identify any of the eight possible genotypes in a single reaction: AA, AE, EE, AS, SC, SS, AC, and CC (A represents wild type allele).

### SPECIFIC REFERENCE

The present application hereby claims benefit of provisional application Ser. No. 60/514,166, filed Oct. 24, 2003.

### GOVERNMENT RIGHTS

This invention was made with the United States Government support under Grant No. 1R43HD37757-01 from the NIH. The United States Government has certain rights in this invention.

### BACKGROUND

The present invention relates to a genotyping method for screening of common mutations within the beta-globin gene. Particularly, what is disclosed is a system and method for beta-globin genotyping using multiplexed melting temperature analysis.

Since its introduction in 1962, newborn screening has been universally accepted with a clear social benefit. Technological advances have played a key role in the rapid development of newborn screening programs around the world. The Guthrie Bacterial Inhibition Assay for phenyalanine was first used for screening of phenylketonura (PKU). Enzyme assays and immunoassays were adapted later for the identification of Congenital Adrenal Hyperplasia, Congenital Hypothyroidism, Cystic Fibrosis, Galactosemia, Biotinidase Deficiency, and Glucose-6-phosphate Dehydrogenase Deficiency. Tandem mass spectrometry was recently adapted to newborn screening, which substantially enhances the screening process and expands coverage to more genetic disorders. There are additional treatable or manageable genetic disorders that meet the World Health Organization and National Academy of Sciences criteria for including in newborn screening program which may potentially be detected by these methodologies. Many other disorders, however, do not. This is not only caused by the limitations of these methodologies, but also to the fact that not all gene products are expressed in blood cells at a detectable level.

Decades of extensive biomedical research activities have provided an understanding of the genetic mechanisms for many inherited disorders. The correlation between phenotype and genotype has been well established for some disorders. In addition, completion of the human genome project will not only allow one to explore more complex human diseases, but will also lead to rapid technological developments in an effort to fully utilize the potential of the vast amounts of genetic information available. All these factors make primary DNA-based screening a very attractive alternative and/or supplement to existing newborn screening methodologies.

Mutation analysis has recently played a very important role as a second-tier confirmatory test in newborn screening. Recent advances in laboratory automation also make population based primary DNA screening feasible and cost effective.

In this work, a high throughput beta-globin genotyping method for newborn screening was developed. The beta-globin gene was chosen for three reasons. First, sickle cell disease and other hemoglobinopathies are part of the mandatory newborn screening program in most U.S. laboratories. Isoelectric focusing electrophoresis is currently used to detect these disorders in many laboratories, and can be used to validate the genotyping method. Second, only three common co-dominant mutations, namely S(A173T), C(G172A), and E(G232A), reach polymorphic frequencies among 750 structural hemoglobin variants. Third, gel electrophoresis methods currently used for detection of hemoglobin disorders are labor intensive, low throughput, and not readily amenable to automation. The new genotyping method is automation friendly, capable of high throughput, and very cost efficient.

### SUMMARY

Genomic DNA is extracted from dried blood collected on a filter paper card preferably using a Beckman Coulter's Biomek FX core robotic system. Fluorescent labeled probes are added to the PCR reaction mixture. Genotyping is achieved through multiplexed melting temperature analysis by a fluorescent resonance energy transfer reaction using a LIGHTTYPER™ instrument in a 384 well plate format. The assay is designed to simultaneously identify eight genotypes, if present, in a single reaction: AA, AE, EE, AS, SS, SC, AC, and CC. The method was validated retrospectively with samples of confirmed genotypes. The method was also prospectively validated with 1,861 samples of unknown genotype screened, in parallel, with isoelectric focusing electrophoresis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows melting peak profiles generated with EE, SS, and CC samples. A yellow line represents melting peaks of an EE sample, which has a wild type peak for the ASC probe set at 56.8° C. and an E mutant peak for the AE probe set at 67° C. A blue line represents melting peaks of a SS sample, which has a S mutant peak for the ASC probe set at 64.5° C. and a wild type peak for the AE probe set at 71.8° C. A red line represents melting peaks of a CC sample, which has a C mutant peak for the ASC probe set at 51° C. and a wild type peak for the AE probe set at 71.8° C.

FIG. 2 shows melting peak profiles for each genotype. A total of 10 previously identified samples for each genotype were used. Each PCR mixture contains both the ASC and AE probe sets. 2A: Peak profile of wild type samples with peaks at 56.8° C. and 71.8° C. 2B: Peak profile of AE samples with peaks at 56.8° C., 67° C., and 71.8° C. 2C: Peak profile of EE samples with peaks at 56.8° C. and 67° C. 2D: Peak profile of AS samples with peaks at 56.8° C., 64.5° C., and 71.8° C. 2E: Peak profile of SS samples with peaks at 67° C. and 71.8° C. 2F: Peak profile of SC samples with peaks at 51° C., 64.5° C., and 71.8° C. 2G: Peak profile of AC samples with peaks at 51° C., 56.8° C., 71.8° C. 2H: Peak profile of CC samples with peaks at 51° C. and 71.8° C. 2I: Visual genotype summary. Samples in column D through column K are AA, AE, EE, AS, SS, SC, AC, and CC, respectively.

FIG. 3 shows melting peak profiles of 384 samples, including 2 AE, 4 AS, and 378 AA samples.

### DETAILED DESCRIPTION OF THE PREFERRED EMBODIMENT

The invention will now be described in detail in relation to a preferred embodiment and implementation thereof which is exemplary in nature and descriptively specific as disclosed. As is customary, it will be understood that no limitation of the scope of the invention is thereby intended. The invention encompasses such alterations and further modifications in the illustrated method, and such further applications of the principles of the invention illustrated herein, as would normally occur to persons skilled in the art to which the invention relates.

Materials and Methods:

1) DNA extraction: Newborn blood is firstly collected. The typical collection method involves placing a droplet of blood obtained from a newborn, for example from a heel prick, on S&S 903 filter paper (Schleicher & Schuell, Keene, N.H.) and sent to a laboratory for routine newborn screening. A disc preferably sized 3/8 inch in diameter is punched from the Dried Blood Spot (DBS) specimen into a 96 well plate for DNA extraction. A robotic system such a a Beckman Coulter Biomek FX core robotic system (Beckman Coulter, Fullerton, Calif.) is used for DNA extraction. The system has a Biomek FX liquid handler, two heat blocks, an automatic plate sealer and plate piercer (Marsh Bio Products, Rochester, N.Y.), and a robotic arm to transport the assay plate between each modular component. The Biomek FX liquid handler adds 30 μl of HPLC-grade methanol into each sample well. The sample plate is transported to the heat blocks for a flexible 15 minutes incubation period at 115° C. to evaporate solvent. The plate is transported back to the liquid handler and 100 μl of 30 mM Tris (pH=8.5) is added to each sample well. The plate is sealed with strong foil using the plate sealer. Genomic DNA is extracted by putting the sealed sample plate on the heat block and incubating for 15 minutes at 115° C. After the sample plate cooled down, it is centrifuged briefly and pierced using the automatic piercer.
2) PCR setup and Cycling condition: PCR primers and fluorescent labeled probe sets (Table 1) were synthesized and HPLC purified by Idaho Technologies, Inc. (Salt Lake City, Utah). The PCR amplification reactions (10 μl) are setup using the Biomek FX core system in a 384 well PCR plate. Each contained 50 mM Tris (pH 9.1), 16 mM ammonium sulfate, 1.5 μg BSA, 3.5 mM MgCl, 200 μM dNTPs, 0.1 μM forward primer, 0.5 μM reverse primer, 0.1 μM of each probe, 0.5 u of Klen Taq polymerase (Ab Pepetides, Inc., St. Louis, Mo.), and 4 μl of extracted DNA. The PCR reaction mixture is covered with 8 μl of mineral oil. PCR is performed in a PrimusHT Multiblock thermal cycler (MWG Biotech, High Point, N.C.). Cycling protocol is one cycle at 94° C. for 1 min; 45 cycles of 94° C. for 20 sec, 60° C. for 30 sec, 72° C. for 20 sec; hold at 72° C. for 1 min and 25° C. for 30 sec; bring temperature to 85° C. at 0.2° C./sec and down to 25° C. at 3° C./sec.
3) Melting temperature analysis: Upon completion of the PCR reaction, the 384 well PCR plate is put into a LIGHTTYPER™ instrument (Roche Diagnostics, Indianapolis, Ind.). The LCD camera exposure time is set at 1000 ms. The plate is then heated, preferably from 40° C. to 85° C. at 0.1° C./sec ramp rate. Melting data is collected and analyzed using the LIGHTTYPER™ Genotyping Software. The genotype is determined for each sample based on the melting profile.
   Results:

This genotyping method is developed based on the fluorescence resonance energy transfer (FRET) reaction. Allelic discrimination is achieved by the difference in melting temperature (ΔTₘ) between the probe set and match or mismatch template. When a probe set hybridizes to a perfectly matched allele, fluorophores on both the detection probe and anchor probe are brought in close proximity, and a FRET reaction occurs. When heated during melting analysis, such proximity will be disrupted, the FRET reaction will be stopped, and a melting curve will be detected. The mid-point of this melting curve is determined and the corresponding temperature is measured as Tₘ. When a probe set hybridizes to a mismatch allele, the close proximity of the two probes will be disrupted at a lower temperature, and a melting curve will be detected at a lower Tₘ.

For beta-globin genotyping, the close proximity of three common mutations, S(A173T), C(G172A), and E(G232A), allows them to be amplified on a single amplicon. Asymmetric PCR was performed to enrich one strand for hybridization. Two probe sets were designed in accordance with Table 1, below.

TABLE 1
Sequences of POR Primers and Probe Sets

| Name | Sequence^{a} |
|---|---|
| Forward | 5′-ACGGCAGAGCCATCTATTGCTTACA-3′ |
| primer | Seq. ID NO: 1 |
| Reverse | 5′-CCAAGAGTCTTCTCTGTCTCCACAT-3′ |
| primer | Seq. ID NO: 2 |
| ASC Anchor | 5′-CAACCTCAAACAGCACCCATGGTGCACCT- |
| Probe | FITC-3′ |
| | Seq. ID NO: 3 |
| ASC | 5′-LC RED 640-CTCCTGTGGAGAAGTCTGC- |
| Detection | OPO3-3′ |
| Probe | Seq. ID NO: 4 |
| AE Anchor | 5′-LC RED 640- |
| Probe | GCAGGTTGGTATCAAGGTTACAAGACAGGTTTAAGGAG- |
| | OPO3-3′ |
| | Seq. ID NO: 5 |
| AE | 5′-GGATGAAGTTGGTCGTCAGGCCCT-FITC-3′ |
| Detection | Seq. ID NO: 6 |
| Probe | |

^{a}LC RED 640 and FITC code for fluorophores and OPO3 codes for phosphate group. ASC probe set perfectly matches to S mutant allele and AE probe set perfectly matches to A (wild type) allele.

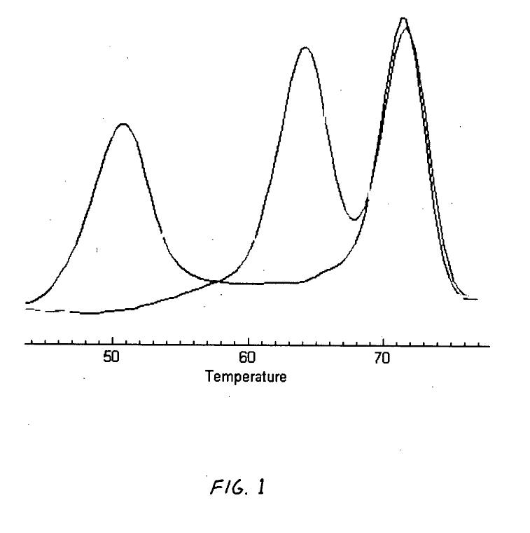

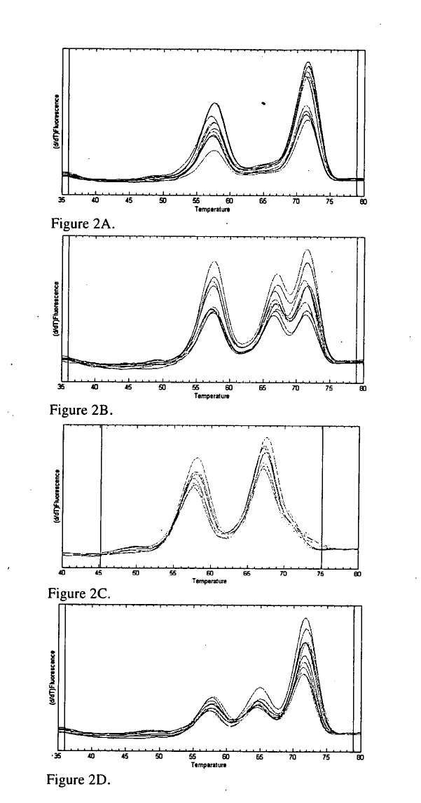

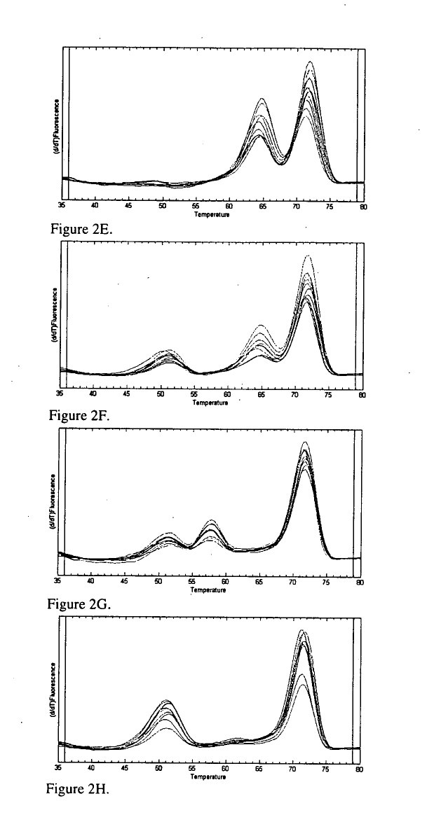

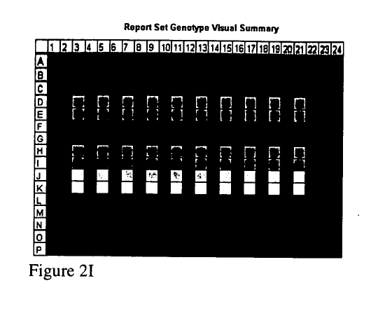

The ASC probe set perfectly matches the S allele, one base pair mismatches the wild type allele, two base pairs mismatch the C allele. The AE probe set perfectly matches the wild type allele, and one base pair mismatches to the E allele. Altogether five distinguishable melting peaks can be detected: a wild type peak for the AE probe set at 71.8° C., an E mutant peak at 67° C.; a S mutant peak at 64.5° C.; a wild type peak for the ASC probe set at 56.8° C.; and, a C mutant peak at 51° C. (FIG. 1). Since both ASC and AE probe can be and in the current assay are present in each sample well, a minimum of two peaks are expected for each sample. To test the specificity of this genotyping method, 10 samples of each genotype were used. These samples were previously identified by isoelectric focusing electrophoresis, and their genotypes were confirmed by second-tier LIGHT CYCLER® assays. The combinations of the five distinct melting peaks result in a unique melting peak profile for each possible genotype. For a wild type sample(s), there are an ASC wild type peak at 56.8° C. and an AE wild type peak at 71.8° C. (FIG. 2A). For an AE sample, there are an ASC wild type peak at 56.8° C., an E mutant peak at 67° C., and an AE wild type peak at 71.8° C. (FIG. 2B). For an EE samples, there are an ASC wild type peak at 56.8° C. and an E mutant peak at 67° C. (FIG. 2C). For an AS sample, there are an ASC wild type peak at 56.8° C., a S mutant peak at 64.5° C., and an AE wild type peak at 71.8° C. (FIG. 2D). For a SS sample, there are a S mutant peak at 64.5° C. and an AE wild type peak at 71.8° C. (FIG. 2E). For a SC sample, there are a C mutant peak at 51° C., a S mutant peak at 64.5° C., and an AE wild type peak at 71.8° C. (FIG. 2F). For an AC sample, there are a C mutant peak at 51° C., an ASC wild type peak at 56.8° C., and an AE wild type peak at 71.8° C. (FIG. 2G). For a CC sample, there is a C mutant peak at 51° C. and an AE wild type peak at 71.8° C. (FIG. 2H). The LIGHTTYPER™ analysis software generates a visual summary of the 384 wells plate with a distinctive color for each genotype (FIG. 2I). The genotyping results were 100% concordant as previously determined for all 80 samples. A set of 8 standard peak profiles was generated from this experiment and will be used for future beta-globin LIGHTTYPER™ genotyping assay.

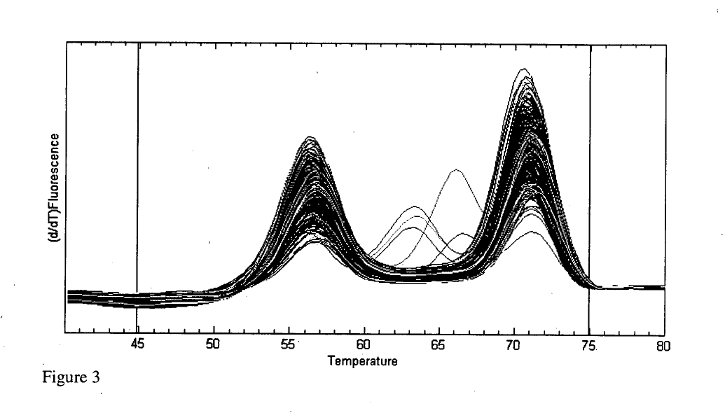

To validate this method, a total of 1,861 unknown samples were screened in parallel by the LIGHTTYPER™ genotyping method and protein isoelectric focusing electrophoresis. As a standard procedure, all positive samples identified by isoelectric focusing are subjected to the second-tier Light Cycler® genotyping assays, which detect the presence of hemoglobin S, C, and E alleles. LIGHTTYPER™ genotyping results obtained from one 384 well plate is shown in FIG. 3. The melting curve profile of each of the 384 samples was compared to the set of 8 standard profiles for automatic genotype interpretation, and a genotype visual summary of the 384 wells plate was generated with a distinctive color for each genotype. Of the 1,861 samples screened, a total of 3 AE, 29 AS, 1 SC, and 8 AC samples were identified by both the LIGHTTYPER™ assays and isoelectric focusing electrophoresis. See Table 2 below:

TABLE 2

| | Isoelectric | | LIGHTTYPER | | |
|---|---|---|---|---|---|
| | Focusing | | Genotyping | | Notes |
| Hemoglobi- | AA: | 1801 | AA: | 1820 | 1. Sample numbers |
| nopathies | AE: | 3 | AE: | 3 | match for all |
| | EE: | 0 | EE: | 0 | calls. |
| | AS: | 29 | AS: | 29 | 2. “others” is |
| | SS: | 0 | SS: | 0 | defined as |
| | SC: | 1 | SC: | 1 | other |
| | AC: | 8 | AC: | 8 | hemoglobin |
| | CC: | 0 | CC: | 0 | variants. |
| | Others: | 3 | Others: | N/A | |

| | | | |
|---|---|---|---|
| Hb Barts | 16 | N/A | Hb Barts is |
| | | | identified as |
| | | | the presence of |
| | | | hemoglobin gamma |
| | | | tetramer due to |
| | | | decreased |
| | | | expression of |
| | | | the α-globin |
| | | | gene. |

Samples with other hemoglobin variants and hemoglobin Barts were only detected by isoelectric focusing but not by the LIGHTTYPER™ assay as expected. Overall, the LIGHTTYPER™ assay shows 100% sensitivity and specificity for genotyping of three common beta-globin mutations S(A173T), C(G172A), and E(G232A).

1. A method for genotyping beta-globin using multiplexed melting temperature analysis, comprising:
extracting genomic DNA samples;
synthesizing PCR primers and fluorescent labeled probes for said samples to form a PCR reaction mixture, wherein said probes are selected from the group consisting of those such sequences as set forth in SEQ ID Nos: 3, 4, 5, and 6; and,
analyzing said PCR reaction mixture to form melting data, wherein a genotype is determined for each said sample based on a melting profile thereof.

2. The method of claim 1, wherein said primers are selected from the group consisting of those such sequences as set forth in SEQ ID NOs: 1 and 2.

3. The method of claim 1, wherein said melting data is obtained while said samples are heated from 40° C. to 85° C.

4. The method of claim 1, further comprising comparing said melting profile to a set of standard profiles for interpreting said genotype.

5. A method for genotyping beta-globin using multiplexed melting temperature analysis, comprising:
extracting genomic DNA samples;
synthesizing PCR primers and fluorescent labeled probes for said samples to form a PCR reaction mixture, wherein said probes are designed to be specific for identifying eight genotypes, if present, in a single reaction; and,
analyzing said PCR reaction mixture to form melting data, wherein said genotypes are determined for each said sample based on a melting profile thereof.

6. The method of claim 5, wherein said probes are selected from the group consisting of those such sequences as set forth in SEQ ID Nos: 3, 4, 5, and 6.

7. The method of claim 5, wherein said melting data is obtained while said samples are heated from 40° C. to 85° C.

8. The method of claim 5, further comprising comparing said melting profile to a set of standard profiles for interpreting said genotypes.


SEQUENCE LISTING (6 sequences)

SEQ ID NO: 1 - DNA, 25 bp, Homo sapiens
agggcagagccatctattgcttaca

SEQ ID NO: 2 - DNA, 25 bp, Homo sapiens
ccaagagtcttctctgtctccacat

SEQ ID NO: 3 - DNA, 29 bp, Homo sapiens
caacctcaaacagcacccatggtgcacct

SEQ ID NO: 4 - DNA, 19 bp, Homo sapiens
ctcctgtggagaagtctgc

SEQ ID NO: 5 - DNA, 38 bp, Homo sapiens
gcaggttggtatcaaggttacaagacaggtttaaggag

SEQ ID NO: 6 - DNA, 24 bp, Homo sapiens
ggatgaagttggtggtgaggccct